SEQUENCE LISTING (4 sequences)

SEQ ID NO: 1 - DNA, 36 bp, Synthetic construct
  source 1..36: mol_type = other DNA
aaaaggtggtggggggggttggtagggtgtcttcta

SEQ ID NO: 2 - DNA, 28 bp, synthetic construct
  source 1..28: mol_type = other DNA
acaggggtgtggggacaggggtgtgggg

SEQ ID NO: 3 - DNA, 15 bp, synthetic construct
  source 1..15: mol_type = other DNA
ggttggtgtggttgg

SEQ ID NO: 4 - DNA, 29 bp, synthetic construct
  source 1..29: mol_type = other DNA
agtccgtggtagggcaggttggggtgact

(19) United States
(12) Patent Application Publication
(10) Pub. No.: US 2025/0130196 A1
(43) Pub. Date: Apr. 24, 2025

(54) Electrochemical Aptamer Sensors with Strong Sensor Response to Large Analytes

(71) Applicant: University of Cincinnati, Cincinnati, OH (US)

(72) Inventor: Jason Heikenfeld, Cincinnati, OH (US)

(73) Assignee: University of Cincinnati, Cincinnati, OH (US)

(21) Appl. No.: 18/835,790

(22) PCT Filed: Feb. 6, 2023
(86) PCT No.: PCT/US23/12443

§ 371 Date: Aug. 5, 2024

Related U.S. Application Data
(60) Provisional application No. 63/306,798, filed on Feb. 4, 2022.

(51) Int. Cl. G01N 27/327; C12N 15/115

(52) U.S. Cl. CPC G01N 27/3277; C12N 15/115; C12N 2310/3517

(57) ABSTRACT

A device and method for detecting the presence of, or measuring the concentration or amount of, at least one large analyte in a sample fluid. The device has at least one electrode, a sample fluid and a plurality of aptamers capable of binding to the analyte. The aptamers are physically bound to the device. In addition, the aptamers each include at least one redox tag and also, the aptamers have two or more binding portions that bind to two or more distinct binding sites on the analyte in the sample fluid.

### CROSS-REFERENCE TO RELATED APPLICATIONS

This application is a continuation of PCT Application No. PCT/US23/12443 filed Feb. 6, 2023, which claims the benefit of the filing date of U.S. Provisional No. 63/306,798 filed Feb. 4, 2022, the disclosure of which is incorporated by reference herein in its entirety.

### FIELD OF THE INVENTION

This invention relates generally to aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte. Aptamers are an example of an affinity-based biosensor.

A major unresolved challenge for aptamer sensors and other affinity-based biosensors is the limit of detection and magnitude of sensor response, especially for measuring larger analytes such a peptide hormones and proteins. In fact, looking at existing demonstrations of electrochemical sensors for proteins you find them often limited to nM to μM detection ranges with <50% change in sensor response, which is far less than where most large analyte are in concentration of pM to nM concentrations and far short of the 100-200% sensor responses that can be achieved for small molecule aptamer based sensors (e.g. see White et al. Langmuir 2008, 24, 18, 10513-10518, or Parolo et al. ACS Sens. 2020, 5, 7, 1877-1881). Aptamers can have improved binding affinity to the analyte of interest and therefore lower limit of detection and a stronger sensor response using techniques such as purposeful mutations, larger aptamer sizes/lengths than exist in aptamer selection libraries, non-native base pairs, aptamer modification, non-traditional tagging approaches, non-DNA portions of an aptamer, and other methods. While aptamer sensors for small molecules are often easily ‘found’ using traditional aptamer selection and adaptation into an electrochemical format, aptamer sensors for proteins suffer from poor performance because in past attempts they were also attempted to be ‘found’ using traditional aptamer selection instead of being ‘built’ with strategies that overcome inherent limitations of aptamer sensors for proteins. Novel approaches for electrochemical aptamer sensors which eliminate the drawbacks of limited binding affinity and sensor response are therefore required.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for detecting or measuring at least one analyte in a sample fluid. The device has at least one electrode, a sample fluid and a plurality of aptamers capable of binding to the analyte. The aptamers are physically bound to the device. In addition, the aptamers each include at least one redox tag and also, the aptamers have two or more binding portions that bind to two or more distinct binding sites on the analyte in the sample fluid. In one embodiment, one or more of the aptamers further include one or more flexible linking portions. In another embodiment, one or more of the aptamers further include one or more substrate linking portions.

In one embodiment, the two or more binding portions provide a binding affinity for the analyte that is at least three times greater than the binding affinity of an aptamer utilizing only one such binding portion. In another embodiment, the two or more binding portions provide a binding affinity for the analyte that is at least ten times greater than the binding affinity of an aptamer utilizing only one such binding portion. In one embodiment, the two or more binding portions provide a binding affinity for the analyte that at least thirty times greater than the binding affinity of an aptamer utilizing only one such binding portion. In another embodiment, the two or more binding portions provide a binding affinity for the analyte that at least one hundred times greater than the binding affinity of an aptamer utilizing only one such binding portion.

In one embodiment, the aptamers are capable of binding to themselves in at least one associative portion of the aptamers. In another embodiment, the aptamers are distally tagged with the redox tag. In one embodiment, the aptamers are internally tagged with the redox tag. In another embodiment, the aptamers are tagged with a plurality of redox tags having different redox potentials.

In one embodiment, the flexible linking portion is a non-native flexible linking portion. In another embodiment, at least one associative portion of the aptamers capable of binding to themselves does so in the absence of the large analyte, and further, wherein the redox tag is in a first distance relative to the electrode. In one embodiment, at least one associative portion of the aptamers capable of binding to themselves disassociates and no longer binds to themselves in the presence of the analyte as it binds to the aptamer, and further, wherein the redox tag is moved to a second distance relative to the electrode that is greater than the first distance relative to the electrode. In another embodiment, >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%. In one embodiment, >25% but no more than 99% of the at least one associative portion of the aptamers are bound to themselves. In another embodiment, >25% but no more than 90% of the at least one associative portion of the aptamers are bound to themselves. In one embodiment, >25% but no more than 50% of the at least one associative portion of the aptamers are bound to themselves.

In another embodiment, the present invention involves a method for measuring one or more analytes in a sample fluid. The method involves first exposing a sample fluid having at least one analyte to at least one electrochemical aptamer-based (EAB) sensor. The sensor has at least one electrode and one or more aptamers capable of binding to the analyte. Also, the aptamers are physically bound to the sensor. In addition, the aptamers are tagged with at least an internal redox tag and a distal redox tag. Secondly, distance redox voltages are measured for the internal redox tag and the distal redox tag. The distance redox voltages are used to calculate ratios of electron transfer currents between the internal redox tag and the distal redox tag.

In one embodiment, one or more of the aptamers also have one or more flexible linking portions. In another embodiment, one or more of the aptamers also have one or more substrate linking portions. In one embodiment, the internal redox tag is methlylene blue. In another embodiment, the distal redox tag is an anthroquinone. In one embodiment, the distal redox tag is ferrocene. In another embodiment, the aptamers are capable of binding to themselves in at least one associative portion of the aptamers.

In one embodiment, at least one associative portion of the aptamers capable of binding to themselves does so in the absence of the large analyte, and further, the redox tag is in a first distance relative to the electrode. In another embodiment, at least one associative portion of the aptamers capable of binding to themselves disassociates and no longer binds to themselves in presence of the analyte as it binds to the aptamer, and further, wherein the redox tag is moved to a second distance relative to the electrode that is greater than the first distance relative to the electrode. In one embodiment, >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%. In another embodiment, >25% but no more than 99% of the at least one associative portion of the aptamers are bound to themselves. In one embodiment, >25% but no more than 90% of the at least one associative portion of the aptamers are bound to themselves.

In another embodiment, the present invention involves a device for detecting or measuring at least one large analyte in a sample fluid. The device has at least one electrode, a sample fluid, and a plurality of aptamers capable of binding to the analyte. The aptamers are physically bound to the device. In addition, the aptamers each include at least one redox tag. Also, the aptamers are capable of binding to themselves in at least one associative portion of the aptamers and do so in the absence of the large analyte. The redox tag is in a first distance relative to the electrode.

In one embodiment, at least one associative portion of the aptamers capable of binding to themselves disassociates and no longer binds to themselves in presence of the analyte as it binds to the aptamer, and further, wherein the redox tag is moved to a second distance relative to the electrode that is greater than the first distance relative to the electrode. In another embodiment, >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%. In one embodiment, >25% but no more than 99% of the at least one associative portion of the aptamers are bound to themselves. In another embodiment, >25% but no more than 90% of the at least one associative portion of the aptamers are bound to themselves. In one embodiment, >25% but no more than 50% of the at least one associative portion of the aptamers are bound to themselves.

### BRIEF DESCRIPTION OF THE DRAWINGS

The objects and advantages of the disclosed invention will be further appreciated in light of the following detailed descriptions and drawings in which:

FIG. 1 is a schematic of a conventional prior art sensor device.

FIG. 2 is a schematic of a device of an embodiment of the present invention.

FIG. 3 is a schematic of a device of an embodiment of the present invention.

FIG. 4 is a schematic of a device of an embodiment of the present invention.

FIG. 5 is a schematic of a device of an embodiment of the present invention.

FIG. 6 is a schematic of a device of an embodiment of the present invention.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of +20% in some embodiments, +10% in some embodiments, +5% in some embodiments, +1% in some embodiments, +0.5% in some embodiments, and +0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “blocking layer” means a homogeneous or heterogeneous layer of material or of one or more types of molecules on an electrode which reduce electrochemical background current and/or current due to electrochemical interference, and which may promote proper freedom of movement for the aptamer which is required for creating a measurable response to analyte concentration.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “large analyte” means an analyte with >3 kDa of molecular weight and in most cases with be >4 kDa such as insulin, BNP or even >10 kDa such as C-reactive protein, IL-6 or other suitable analytes.

As used herein, the term “substrate linking portion of an aptamer (SLPA)” means a subset of an aptamer whose specific purpose at least includes linking the aptamer to the substrate and/or properly spacing or positioning the rest of the aptamer relative the substrate. Example substrate linking portions are thiol linkage to a gold electrode substrate, phosphate or silane linkage to oxide substrates, or other suitable linkage/substrate combinations.

As used herein, the term “first binding portion of an aptamer (FBPA)” means a subset of an aptamer with multiple portions that bind to the analyte of interest, whose position along the aptamer as closest to the SLPA compared to other binding portions of the aptamer. The FBPA may encompass in part or entirety the functions of the SLPA.

As used herein, the term “second binding portion of an aptamer (SBPA)” means a subset of an aptamer with multiple portions that bind to the analyte of interest, whose position along the aptamer is second closest to the SLPA compared to other binding portions of the aptamer. A third, fourth, or any plurality of binding portions of an aptamer are also possible.

As used herein, the term “flexible linking portion of an aptamer (FLPA)” means a subset of an aptamer whose specific purpose at least includes linking at least two binding portions of an aptamer such as a FBPA and SBPA. Depending on its needed length the FLPA may encompass in part functions of a binding site. A FLPA is not always required but in most instances is required because binding sites are not typically immediately adjacent to each other and because physical flexibility is often needed between the FBPA and SBPA. An example FLPA is a chain of thymine bases.

As used herein, the term “non-native FLPA” means a FLPA that is comprised of a material other than DNA. Examples include phosphoramidite for conjugation of oligonucleotides with biomolecules but instead in this use case to link a FBPA and an SBPA, using for example polyethylene glycol phosphoramidite. Non-native FLPAs can further provide greater physical flexibility than a FLPA.

As used herein, a “two or more binding portions” or a “plurality of binding portions” for an aptamer means that each binding portion individually would bind to the target analyte with the additional requirement that each binding portion is separated by at least one connection of molecules, the connection of molecules not binding to the target analyte during binding of the aptamer to the target analyte. For example: a FBPA linked to a FLPA linked to a SBPA is an aptamer with “two or more binding portions”.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Redox tags can be tagged at the end of an aptamer or internally along the aptamer using for example thymine base modification, referred to as ‘distal tagging’ and ‘internal tagging”, respectively.

As used herein, the term “change in electron transfer” means a redox molecule whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, “redox tag current” is the amplitude of the faradaic redox tag peak current minus the background current amplitude outside the redox peak in a given voltammetric scan.

As used herein, “normalized redox-tag current” is the redox-tag current normalized to the first measurement taken.

As used herein, “background current” is the voltammetric current that would be measured if the aptamer molecules were not tagged with a redox reporter including, for example, capacitive currents and competing redox processes such as oxygen reduction.

As used herein, “adjusted current” is the combined redox tag current and background current of a square-wave voltammogram adjusted such that the minimum current is set to 0 A in the presentation of the voltammogram such that voltammograms can be plotted side by side and compared with greater ease.

As used herein, “sensor response” is the change in redox tag current due to binding of the target analyte to the aptamer, also known as signal gain, which can either increase or decrease based on the aptamer and the voltammetric time scale. Sensor response may also applied to alternative measures such as amperometry or chronoamperometry or other approaches which do not measure a voltammogram. As used herein “change in sensor response” is the percentage change in the sensor response in response to increasing analyte concentration compared to sensor response if no analyte were present. For example, if the peak redox tag current was 1 μA with no analyte present and adding analyte caused the peak redox tag current to be 0.5 or 1.5 μA then the change in sensor response would be −50% sensor off response or +50% sensor on response respectively.

As used herein, the term “sensing monolayer” means at least a plurality of aptamers on a working electrode, which may also include a plurality of molecules or mixtures of molecules that form a blocking layer or an anti-fouling layer.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “continuous sensing” simply means the device records a plurality of readings over time. Even a point-of-care testing device which provides a single data point can be considered a continuous sensing device if, for example, it is a 15 minute test, that operates by taking multiple data points over 15 minutes and averaging them to provide a single data measure.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

In one embodiment, the present invention involves a device for detecting or measuring at least one analyte in a sample fluid. The device has at least one electrode, a sample fluid and a plurality of aptamers capable of binding to the analyte. The aptamers are physically bound to the device. In addition, the aptamers each include at least one redox tag and also, the aptamers have two or more binding portions that bind to two or more distinct binding sites on the analyte in the sample fluid. In one embodiment, one or more of the aptamers further include one or more flexible linking portions. In another embodiment, one or more of the aptamers further include one or more substrate linking portions.

In one embodiment, the two or more binding portions provide a binding affinity for the analyte that is at least three times greater than the binding affinity of an aptamer utilizing only one such binding portion. In another embodiment, the two or more binding portions provide a binding affinity for the analyte that is at least ten times greater than the binding affinity of an aptamer utilizing only one such binding portion. In one embodiment, the two or more binding portions provide a binding affinity for the analyte that at least thirty times greater than the binding affinity of an aptamer utilizing only one such binding portion. In another embodiment, the two or more binding portions provide a binding affinity for the analyte that at least one hundred times greater than the binding affinity of an aptamer utilizing only one such binding portion.

In one embodiment, the aptamers are capable of binding to themselves in at least one associative portion of the aptamers. In another embodiment, the aptamers are distally tagged with the redox tag. In one embodiment, the aptamers are internally tagged with the redox tag. In another embodiment, the aptamers are tagged with a plurality of redox tags having different redox potentials.

In another embodiment, the present invention involves a method for measuring one or more analytes in a sample fluid. The method involves first exposing a sample fluid having at least one analyte to at least one electrochemical aptamer-based (EAB) sensor. The sensor has at least one electrode and one or more aptamers capable of binding to the analyte. Also, the aptamers are physically bound to the sensor. In addition, the aptamers are tagged with at least an internal redox tag and a distal redox tag. Secondly, distance redox voltages are measured for the internal redox tag and the distal redox tag. The distance redox voltages are used to calculate ratios of electron transfer currents between the internal redox tag and the distal redox tag.

In one embodiment, one or more of the aptamers also have one or more flexible linking portions. In another embodiment, one or more of the aptamers also have one or more substrate linking portions. In one embodiment, the internal redox tag is methlylene blue. In another embodiment, the distal redox tag is an anthroquinone. In one embodiment, the distal redox tag is ferrocene. In another embodiment, the aptamers are capable of binding to themselves in at least one associative portion of the aptamers.

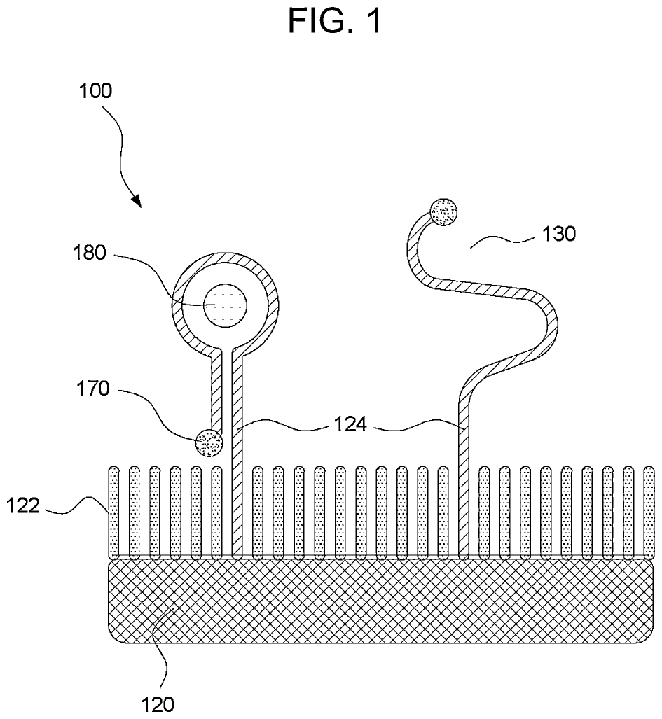

With reference to FIG. 1, a conventional prior art sensor device 100 as placed initially in a sample fluid 130 such as interstitial fluid is shown, comprising: at least one working electrode 120 such as gold, carbon, or other suitable electrode material; at least one blocking or protective layer 122 of a plurality of molecules such as mercaptohexanol or hexanethiol that are thiol bonded to the electrode, or a plurality of natural solutes in blood that can act as a blocking layer, or other suitable molecules depending on application and on the choice of electrode 120 material; at least one aptamer 124 that is linked to the gold 120 via a thiol bond or other suitable bond to an electrode 120 or to a blocking layer 122. The aptamer 124 is responsive to binding to an analyte 180 and which contains a redox tag 170 such as methylene blue. In the generic example taught for FIG. 1, the aptamer 124 is a simple stem loop (hairpin) aptamer where analyte 180 binding causes the stem loop to form and the redox tag current measured from the redox tag 170 to increase, as measured using square wave voltammetry or other suitable technique. The sensor can also have more than one stem loop, for example such as the cocaine aptamer which has >100% sensor response to cocaine as taught in White et al. Langmuir 2008, 24, 18, 10513-10518. In the absence of analyte 180 binding to the aptamer 124, the stem loop is broken and the redox current would decrease. Thus, a measurement of electrical current can be used to interpret changes in the concentration of the analyte 180. The majority of electrochemical aptamer sensors use ‘signal ON’ motifs like that illustrated in FIG. 1 which produce large sensor responses of >100% by leveraging a stem loop or other stable configuration which brings the redox tag 170 close to the electrode 120 in a stable geometry with significantly increased redox tag current. These ‘signal ON’ geometries use a single binding domain of the aptamer 124 to the analyte 180 because the analyte is a small molecule and because the small molecule is at high concentrations such that single binding domains can be sufficient. Making a similar sensor for a larger peptide or protein or other analyte such as insulin, BNP, c-reactive protein, IL-6 or other analytes is much more difficult because they are at much lower concentrations (pM to nM range) and because they are so large that a single binding domain will have difficulty in many cases binding to the analyte with a binding affinity that is close to the physiologically expected concentrations in a biofluid such as interstitial fluid.

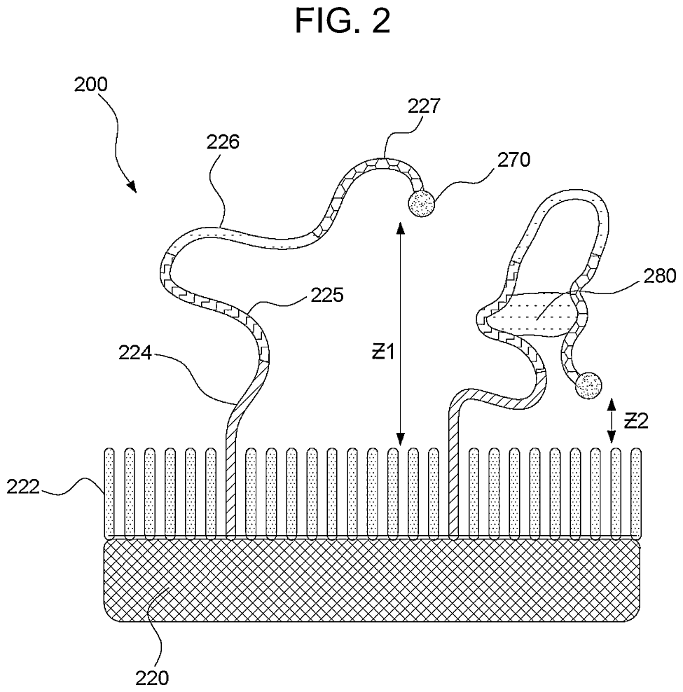

With reference to FIG. 2, where like numerals refer to like features, in an embodiment of the present invention a device 200 having an electrode 220 and a blocking layer 222 utilizes an aptamer comprised of elements 224, 225, 226, and 227. Element 224 can be, for example, a substrate linking portion of an aptamer (SLPA). Element 225 can be, for example, a first binding portion of an aptamer (FBPA). Element 226 can be, for example, a flexible linking portion of an aptamer (FLPA) or a non-native FLPA, and element 227 can be a second binding portion of an aptamer (SBPA) which includes a distally positioned redox tag 270 such as methylene blue. The specific geometries, lengths, and other features of FIG. 2 and other embodiments should only be limited as specifically specified herein, as there are multiple stable, semi-stable, and freely moving geometries possible for aptamer design. FIG. 2 will now be taught in greater detail. When Element 224 is an SLPA, it can be a variety of sequences or materials as used in conventional electrochemical aptamer-based sensors and its length and flexibility optimized for a given analyte 280. When Element 225 is an FBPA, it can be a variety of sequences or materials as used for example in aptamers or other linkers used in biosensor chemistry, including for example a chain of thymine bases, or for example non-native chains of polyethylene glycol. When Element 225 is a FBPA, and Element 227 is an SBPA, they are chosen or designed to bind to distinct portions of the large analyte 280 inducing a change in availability of the redox tag 270 for electron transfer with electrode 220. This change in availability, for simplicity of illustration, is depicted as a change in distance Z1, Z2, as labeled in FIG. 2, and the exact nature of change in electron transfer can of course be more complicated as understood by those skilled in the art of electrochemical aptamer-based sensors. The device 200, could use a combination of FBPA and SBPA portions to achieve a lower limit of detection and overall binding affinity than what either a FBPA portion or a SBPA portion could achieve alone. For example, insulin is a high-value large analyte target for which it is difficult to make a robust binding aptamer with a single binding site (e.g. FIG. 1) that measured insulin at physiological concentrations. Two or more binding sites (a plurality) and two or more binding portions of the aptamer (FBPA, SBPA, etc., a plurality) in combination lowers the limit of detection by increasing the overall binding affinity by, in alternate embodiments, at least 3×, 10×, 30×, 100×, or more. Examples of binding portions of aptamers can be derived from new aptamer selection or existing aptamer libraries for the large analyte insulin, such as those taught by White and colleagues in ACS Sens. 2019, 4, 498-503, ‘Electrochemical Aptamer-Based Sensor for Real-Time Monitoring of Insulin’. For example, the SLPA portion and FBPA portion may be 5′-HS-C6-SEQ.1, where SEQ. 1 is:

SEQ 1: AAAAGGTGGTGGGGGGGGTTGGTAGGGTGTCTTCTA

A FLPA can then include a plurality of thymine nucleotides.

The SBPA can then be for example from Jennifer Y. Gerasimov, Cody S. Schaefer, Weiwei Yang, Rebecca L. Grout, Rebecca Y. Lai, ‘Development of an electrochemical insulin sensor based on the insulin-linked polymorphicregion’ Biosensors and Bioelectronics, Volume 42, 2013, Pages 62-68, ISSN 0956-5663

SEQ 2: ACAGGGGTGTGGGGACAGGGGTGTGGGG

One of the first demonstrations ever for an electrochemical aptamer biosensor with a single binding site (e.g. FIG. 1), was for the large analyte molecule Thrombin as taught by Plaxco and colleagues in Angew. Chem. Int. Ed. 2005, 44, 5456-5459, ‘Label-free electronic detection of thrombin in blood serum by using an aptamer-based sensor’. Thrombin may also serve as an example application of the present invention by utilizing Sequence 3 and Sequence 4 for either a FBPA portion or a SBPA portion.

SEQ 3: GGTTGGTGTGGTTGG
SEQ 4: AGTCCGTGGTAGGGCAGGTTGGGGTGACT

For any large analyte the specific choice of FBPA, SBPA, optional SLPA and FLPA, are those that typically will provide two important performance parameters: (1) a languire-isotherm binding response curve centered around the desired concentrations to be measured for large analyte 280; (2) a large or maximum change in redox tag electron transfer as large analyte 280 binds to the aptamer portions such as 225 and 227. Although not specifically shown herein, a third binding site, binding portion of the aptamer, additional FLPAs, or even more, may be utilized, referred to as a plurality of binding sites and binding portions of the aptamer.

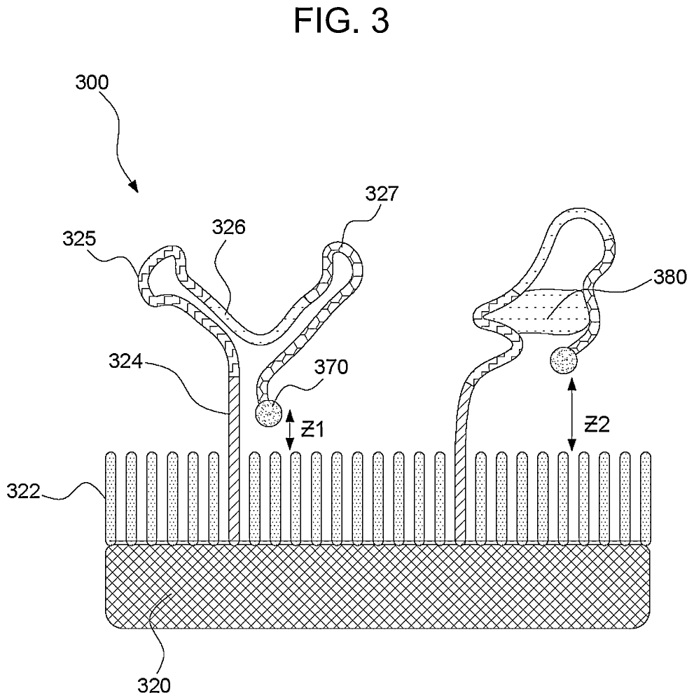

With reference to FIG. 3, where like numerals refer to like features, in an embodiment of the present invention a device 300 having an electrode 320 and a blocking layer 322 may utilize an aptamer comprised of elements 324, 325, 326, 327 that has structure with or without the binding of analyte 380. A partially or fully complimentary sequence scan be used to form stem-loop (‘hairpin’) or other structures that aid in optimizing the performance of the device 300, for example element 325 is associated with one stem loop and element 327 with the other stem loop. Element 324 can be, for example, a substrate linking portion of an aptamer (SLPA). Element 325 can be, for example, a first binding portion of an aptamer (FBPA). Element 326 can be, for example, a flexible linking portion of an aptamer (FLPA) that is also designed to be complimentary in nucleotide bases to support stem loop formation, and element 327 can be a second binding portion of an aptamer (SBPA) which includes a distally positioned redox tag 370 such as methylene blue. Z1 and Z2 indicate a change in availability of the redox tag 370 for electron transfer with electrode 320 as discussed in FIG. 2.

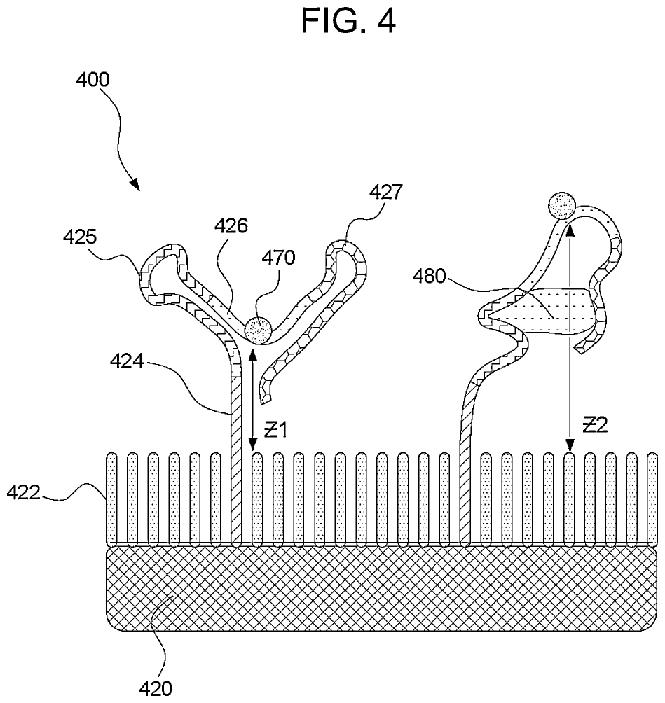

With reference to FIG. 4, where like numerals refer to like features, in an embodiment of the present invention a device 400 having an electrode 420 and a blocking layer 422 may utilize internal tagging of an aptamer comprised of elements 424, 425, 426, 427. When an aptamer is designed with two or more binding portions, its length can go beyond the total length that provides robust performance for an electrochemical aptamer-based sensor (e.g. the distal tagging strategy would provide no discernable or poor sensor response with binding to analyte 480). Internal tagging, as illustrated in the example of FIG. 4 (see redox tag 470), may in some cases result in a larger change in electron transfer with analyte binding. Element 424 can be, for example, a substrate linking portion of an aptamer (SLPA). Element 425 can be, for example, a first binding portion of an aptamer (FBPA). Element 426 can be, for example, a flexible linking portion of an aptamer (FLPA), and element 427 can be a second binding portion of an aptamer (SBPA). In this example, element 426 includes an internally positioned redox tag 470 such as methylene blue. Z1 and Z2 indicate a change in availability of the redox tag 470 for electron transfer with electrode 420 as discussed in FIG. 2

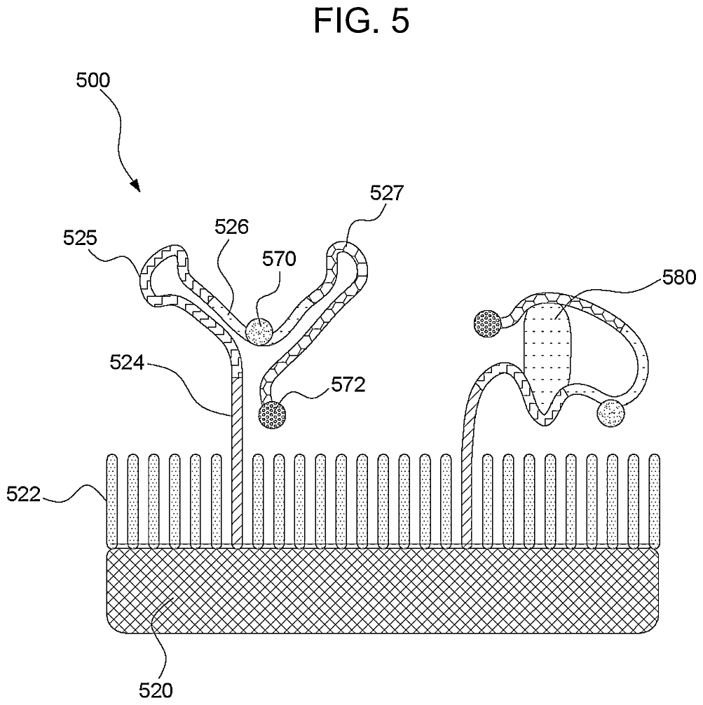

With reference to FIG. 5, where like numerals refer to like features, in an embodiment of the present invention a device 500 having an electrode 520 and a blocking layer 522 is tagged into two distinct locations of the aptamer (comprised of elements 524, 525, 526, 527), such as for example internal and distal tagging. As a result of binding to the large analyte 580, one or more of the electron transfer rates will change from either redox tag 570 or 572, allowing a calibration-free or stronger measurement of the analyte 580 binding. For example, in a non-limiting example such as that illustrated in FIG. 5, a first redox tag 570 such as methlylene blue is internally tagged and a second redox tag 572 such as ferrocene or some other anthroquinone redox tag is tagged distally, redox tags 570 and 572 have distance redox voltages such that they are distinguishable on a voltammogram or other measurement technique, and their availability for electron transfer changes oppositely with analyte binding enabling the ratios of their electron transfer currents to be measured instead of absolute magnitude of current or just change in current. Not only does this increase the overall strength and accuracy of measurement but it could enable calibration free operation. Element 524 can be, for example, a substrate linking portion of an aptamer (SLPA). Element 525 can be, for example, a first binding portion of an aptamer (FBPA). Element 526 can be, for example, a flexible linking portion of an aptamer (FLPA), and element 527 can be a second binding portion of an aptamer (SBPA).

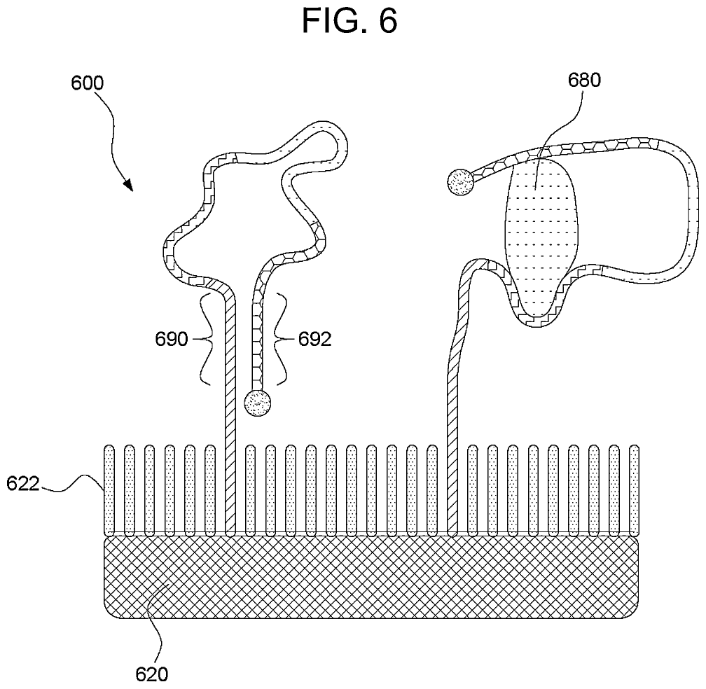

In yet another embodiment of the present invention, the sensors such as 400, 500, or other sensors operating as taught herein can have a strong signal off response where the redox tag current decreases as large analyte binding occurs to the aptamer. Examining existing demonstrations of electrochemical sensors for large analytes one routinely sees a <50% change in total sensor response over the full range of analyte concentrations the sensor responds to, which is far short of the 100-200% sensor responses that can be achieved for small molecule aptamer based sensors (e.g. see White et al. Langmuir 2008, 24, 18, 10513-10518, or Parolo et al. ACS Sens. 2020, 5, 7, 1877-1881). This is in part because these aptamer sensors do not switch like the aptamer sensor described in FIG. 2, which goes from an unbound (distanced redox tag) to a bound stem-loop configuration where the change in redox tag distance from the electrode is very small and held stable in that configuration. The present invention further enables >100% sensor response to large analytes by employing aptamer geometry changes and changes in redox tag current as taught for example in FIGS. 4 and 5, or in single STEM loop, g-quadraplex, or other aptamer geometries that enable >100% sensor response to a large analyte. Such a large change in sensor response for this embodiment of the present invention requires complimentary base pairing between two or more sections of the aptamer that forms a geometry such as the stem of a stem loop configuration. Complimentary base pairing extends to any type of chemically modified or molecule that satisfies the general principles taught for the present invention and is not specifically limited to DNA bases. However, DNA bases can be used as an example. For example, as illustrated in FIG. 6, in a sensor 600 having an electrode 620 and a blocking layer 622, the redox tag can be forced into a state of close proximity to the electrode 620 by virtue of one or more complimentary base pairings or ‘associative portions’ such as a stem that is in close proximity to the electrode for example with a first portion of the stem 690 and a second portion of the stem 692 that are complimentary. The aptamer of FIG. 6 may or may not require FBPA, SBPA or FLPA segments. For example, the first portion of the stem 690 could be CGG and the second portion of the stem 692 GCC. For example, the first portion of the stem 690 could be CGGCATG and the second portion of the stem 692 could be GCCGTG. For example, the first portion of the stem 690 could be CGGCATGGG and the second portion of the stem 692 could be GCCGTGCCC. Even larger stems are possible. The longer the compliment length the more likely the aptamer is to have a strong signal without large analyte. However, if the stem 690, 692 association is too strong then the aptamer will not change geometry during large analyte binding to break the stem 690, 692 and move the redox tag 670 far away from the electrode 620 for a signal off response that is >100%. This requires an optimal associative strength or energy for the stem 690, 692 that is neither too strong nor too weak, because if the associated strength or energy is too strong the binding affinity of the large analyte 680 to the aptamer will increase (e.g. from a binding affinity or Kd of 100 pM to for example 2 nM which would not allow detection for example of a low concentration large analyte such as BNP or insulin). This is can be parametrically specified as follows for a wearable sensor inserted into the human dermis: in serum or interstitial fluid at 33 Celsius and with no large analyte 680 present >25% but no more than 99.9% of the aptamers form this stem 690, 692 and a when large analyte 680 is added a sensor off response of >100% is obtained as the stem 690, 692 is broken and the redox tag 670 distanced from the electrode 620. In one embodiment, this can be described as follows: in serum or interstitial fluid at 33 Celsius and with no large analyte 680 present >25% but no more than 99% of the aptamers form this stem 690, 692 and a when large analyte 680 is added a sensor off response of >100% is obtained as the stem 690, 692 is broken and the redox tag 670 distanced from the electrode. In another embodiment, this can be described as follows: in serum or interstitial fluid at 33 Celsius and with no large analyte 680 present >25% but no more than 90% of the aptamers form this stem 690, 692 and a when large analyte 680 is added a sensor off response of >100% is obtained as the stem 690, 692 is broken and the redox tag 670 distanced from the electrode 620. In another embodiment, >25% but no more than 50% of the aptamers form this stem 690, 692 when the device is in serum or interstitial fluid at 33° Celsius and with no large analyte 680 present. When large analyte 680 is added, a sensor off response of >100% is obtained as the stem 690, 692 is broken and the redox tag 670 is distanced from the electrode 620.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting or measuring at least one large analyte in a sample fluid, the device comprising:
at least one electrode;
a sample fluid; and
a plurality of aptamers capable of binding to the analyte, wherein the aptamers are physically bound to the device;
wherein the aptamers each include at least one redox tag and further, wherein the aptamers have two or more binding portions that bind to two or more distinct binding sites on the analyte in the sample fluid.

2. The device of claim 1, wherein one or more of the aptamers further comprise one or more flexible linking portions.

3. The device of claim 1, wherein one or more of the aptamers further comprise one or more substrate linking portions.

4. The device of claim 1, wherein the two or more binding portions provide a binding affinity for the large analyte that is at least three times greater than the binding affinity of an aptamer utilizing only one such binding portion.

5. The device of claim 1, wherein the two or more binding portions provide a binding affinity for the large analyte that is at least ten times greater than the binding affinity of an aptamer utilizing only one such binding portion.

6. The device of claim 1, wherein the two or more binding portions provide a binding affinity for the large analyte that is at least thirty times greater than the binding affinity of an aptamer utilizing only one such binding portion.

7. The device of claim 1, wherein the two or more binding portions provide a binding affinity for the large analyte that is at least one hundred times greater than the binding affinity of an aptamer utilizing only one such binding portion.

8. The device of claim 1, wherein the aptamers are capable of binding to themselves in at least one associative portion of the aptamers.

9. The device of claim 1, wherein the aptamers are distally tagged with the redox tag.

10. The device of claim 1, wherein the aptamers are internally tagged with the redox tag.

11. The device of claim 1, wherein the aptamers are tagged with a plurality of redox tags having different redox potentials.

12. The device of claim 1, where the flexible linking portion is a non-native flexible linking portion.

13. The device of claim 8, wherein at least one associative portion of the aptamers capable of binding to themselves does so in the absence of the large analyte, and further, wherein the redox tag is in a first distance relative to the electrode.

14. The device of claim 13, wherein at least one associative portion of the aptamers capable of binding to themselves disassociates and no longer binds to themselves in the presence of the analyte as it binds to the aptamer, and further, wherein the redox tag is moved to a second distance relative to the electrode that is greater than the first distance relative to the electrode.

15. The device of claim 14, wherein >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%.

16. The device of claim 15, wherein >25% but no more than 99% of the at least one associative portion of the aptamers are bound to themselves.

17. The device of claim 15, wherein >25% but no more than 90% of the at least one associative portion of the aptamers are bound to themselves.

18. The device of claim 15, wherein >25% but no more than 50% of the at least one associative portion of the aptamers are bound to themselves.

19. A method for measuring one or more analytes in a sample fluid, comprising;
a. exposing a sample fluid having at least one large analyte to at least one electrochemical aptamer-based (EAB) sensor, wherein the sensor comprises at least one electrode and one or more aptamers capable of binding to the large analyte, wherein the aptamers are physically bound to the sensor; and wherein the aptamers are tagged with at least a first internal redox tag and a second redox tag at a different location, the redox tags having different redox voltages;
b. measuring redox currents for the first internal redox tag and the second redox tag;
c. using the distance redox currents to calculate ratios of electron transfer currents between the first internal redox tag and the second redox tag.

20. The method of claim 19, wherein one or more of the aptamers further comprise one or more flexible linking portions.

21. The method of claim 19, wherein one or more of the aptamers further comprise one or more substrate linking portions.

22. The method of claim 19, wherein only one of the redox tags on each aptamer is methlylene blue.

23. The method of claim 19, wherein only one of the redox tags on each aptamer is an anthroquinone.

24. The method of claim 19, wherein only one of the redox tags on each aptamer is ferrocene.

25. The method of claim 19, wherein the aptamers are capable of binding to themselves in at least one associative portion of the aptamers.

26. The method of claim 25, wherein at least one associative portion of the aptamers capable of binding to themselves does so in the absence of the large analyte, and further, the redox tag is in a first distance relative to the electrode.

27. The method of claim 25, wherein at least one associative portion of the aptamers capable of binding to themselves disassociates and no longer binds to themselves in presence of the analyte as it binds to the aptamer, and further, wherein the redox tag is moved to a second distance relative to the electrode that is greater than the first distance relative to the electrode.

28. The method of claim 26, wherein >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%.

29. The method of claim 28, wherein >25% but no more than 99% of the at least one associative portion of the aptamers are bound to themselves.

30. The method of claim 28, wherein >25% but no more than 90% of the at least one associative portion of the aptamers are bound to themselves.

31. A device for detecting or measuring at least one large analyte in a sample fluid, the device comprising:
at least one electrode;
a sample fluid; and
a plurality of aptamers capable of binding to the analyte, wherein the aptamers are physically bound to the device;
wherein the aptamers each include at least one redox tag and further, wherein the aptamers are capable of binding to themselves in at least one associative portion of the aptamers and do so in the absence of the large analyte, and further, wherein the redox tag is in a first distance relative to the electrode.

32. The device of claim 31, wherein at least one associative portion of the aptamers capable of binding to themselves disassociates and no longer binds to themselves in presence of the analyte as it binds to the aptamer, and further, wherein the redox tag is moved to a second distance relative to the electrode that is greater than the first distance relative to the electrode.

33. The device of claim 32, wherein >25% but no more than 99.9% of the at least one associative portion of the aptamers are bound to themselves in the following conditions: in serum or interstitial fluid at 33° Celsius with no large analyte present, and further, when large analyte is added and binds to the aptamer the sensor off response is >100%.

34. The device of claim 33, wherein >25% but no more than 99% of the at least one associative portion of the aptamers are bound to themselves.

35. The device of claim 33, wherein >25% but no more than 90% of the at least one associative portion of the aptamers are bound to themselves.

36. The device of claim 33, wherein >25% but no more than 50% of the at least one associative portion of the aptamers are bound to themselves.
